(19) United States
(12) Patent Application Publication
(10) Pub. No.: US 2014/0336747 A1
(43) Pub. Date: Nov. 13, 2014

(54) BIORESORBABLE IMPLANTS FOR TRANSMYOCARDIAL REVASCULARIZATION

(71) Applicant: Abbott Cardiovascular Systems Inc., Santa Clara, CA (US)

(72) Inventors: Richard Rapoza, San Francisco, CA (US); Nadine Ding, San Jose, CA (US); Yunbing Wang, Sunnyvale, CA (US); Stephen Pacetti, San Jose, CA (US); Derek Mortisen, Palo Alto, CA (US)

(21) Appl. No.: 14/253,760

(22) Filed: Apr. 15, 2014

Related U.S. Application Data
(60) Provisional application No. 61/812,651, filed on Apr. 16, 2013.

(51) Int. Cl. A61F 2/82; A61L 29/16; A61L 29/14

(52) U.S. Cl. CPC A61F 2/82; A61L 29/146; A61L 29/148; A61L 29/16
USPC 623/1.15; 623/1.1; 623/1.39; 623/1.38; 623/1.43

(57) ABSTRACT

Implants for treating insufficient blood flow to a heart muscle with transmyocardial revascularization are disclosed. Methods of treating insufficient blood flow to a heart muscle with the implant are also disclosed. The implant can have a body with an inner lumen that supports a channel in the heart muscle to allow for increased blood flow through the lumen upon implantation. The implant can include active agents to prevent or inhibit thrombotic closure of the channel, to promote vascularization, or both.

This application claims the benefit of U.S. Application Ser. No. 61/812,651 filed on Apr. 16, 2013, which is incorporated by reference herein.

### BACKGROUND OF THE INVENTION

1. Field of the Invention

This invention relates to bioresorbable implants and methods of using such implants for treatments of refractory angina and ischemic or infarcted myocardium involving transmyocardial revascularization.

2. Description of the State of the Art

This invention relates generally to treatment of ischemic and infarcted myocardium resulting from coronary heart disease with endoprostheses that are adapted to be implanted in the myocardium to improve blood flow to the heart. An “endoprosthesis” corresponds to an artificial device that is placed inside the body.

Patients with coronary artery disease are treated with percutaneous interventional procedures (angioplasty and stenting), coronary artery bypass grafting (surgery) and medications to improve blood flow to the heart muscle. In particular, stents are generally cylindrically shaped devices that function to hold open and sometimes expand a segment of a blood vessel or other anatomical lumen such as urinary tracts and bile ducts. A “lumen” refers to a cavity of a tubular organ such as a blood vessel. Stents are often used in the treatment of atherosclerotic stenosis in blood vessels, where “stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffold gets its name because it physically holds open and, if desired, expands the wall of a passageway in a patient. Typically, stents are capable of being compressed or crimped onto a catheter to a reduced diameter so that they can be delivered to and deployed at a treatment site.

For some patients, the above-mentioned treatments for coronary heart disease are not appropriate. For example, the patient's condition may have progressed to the point that the above interventional procedures would not work or were attempted and were not effective. In addition, by-pass surgery and medication alone is inadequate to treat the condition. Such procedures may not eliminate the symptoms of chest pain, also called angina, typically experienced by patients with coronary heart disease. Specifically, angina is pain, “discomfort,” or pressure localized in the chest that is caused by an insufficient supply of blood (ischemia) to the heart muscle. It is also sometimes characterized by a feeling of choking, suffocation, or crushing heaviness in the chest region.

Transmyocardial revascularization (TMR) is an alternative procedure for patients with ischemic or hibernating myocardium resulting from coronary artery disease. TMR is a treatment aimed at improving blood flow to areas of the heart that can no longer be treated by angioplasty or surgery. TMR is a surgical procedure in which small channels are created in the heart muscle with a laser. The channels are intended to improve blood flow in the heart. The procedure is performed through a small left chest incision or through a midline incision. Frequently, it is performed with coronary artery bypass surgery, but occasionally it is performed independently.

Once the incision is made, the surgeon exposes the epicardial surface of the left ventricle. A laser handpiece is then positioned on the area of the heart to be treated. A special high-energy, computerized carbon dioxide (CO₂) laser, called the CO₂ Heart Laser 2, is used to create between 20 to 40 one-millimeter-wide channels (about the width of the head of a pin) in the ischemic or oxygen-poor region of the left ventricle (left pumping chamber) of the heart. The doctor determines how many channels to create during the procedure. The outer areas of the channels close, but the inside of the channels remain open inside the heart to improve blood flow. A computer is used to direct the CO₂ laser beams to the appropriate area of the heart in between heartbeats, when the ventricle is filled with blood and the heart is relatively still. This helps to prevent electrical disturbances in the heart.

Clinical evidence suggests blood flow is improved in two ways: (1) the channels act as bloodlines, when the ventricle pumps or squeezes oxygen-rich blood out of the heart, it sends blood through the channels, restoring blood flow to the heart muscle; (2) the procedure may promote angiogenesis, or growth of new capillaries (small blood vessels) that help supply blood to the heart muscle. Another proposed mechanism of benefit is denervation of the myocardium with a resulting decrease in angina symptoms.

Maintaining blood flow through the ventricle and revascularization are critical aspects of the procedure.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if each said individual publication, patent, or patent application was fully set forth, including any figures, herein.

### SUMMARY OF THE INVENTION

The first embodiments of the present invention include a method of treating insufficient blood flow to a heart muscle comprising: creating a channel in a heart muscle of a patient in need of increased blood flow to the heart muscle due to insufficient blood flow to the heart muscle; disposing an implant within the channel; wherein the implant supports and maintains at least a portion of the channel to allow oxygen rich blood to flow through the channel.

The first embodiments may have one or more, or any combination of the following aspects (1)-(8): (1) wherein the implant comprises a shape defined by a wall that encloses a cavity or lumen; (2) wherein the implant comprises a tubular body comprising walls surrounding a lumen through which the blood flows; (3) wherein the implant comprises a tubular body comprising walls surrounding a lumen, wherein the walls are fenestrated, porous, contain pores, or have open cells; (4) wherein the implant is bioresorbable and completely resorbs away from the channel after providing the support to the portion of the channel; (5) wherein the implant comprises a bioresorbable polymer; (6) wherein the implant comprises a tubular body, the method further comprising radially expanding the tubular body after being disposed in the channel to an outer diameter larger than a diameter of the channel which provides for increased blood flow; (7) wherein the channel extends from a ventricle through the heart muscle or myocardium to the pericardium to allow oxygen-rich blood to flow into the channel; and (8) wherein the channel is formed from an endocardial or ventricle side of the heart and the implant is delivered percutaneously into the channel from the ventricle into the myocardium.

The second embodiments of the present invention include a method of treating insufficient blood flow to a heart muscle comprising: creating a channel in a heart muscle of a patient in need of increasing blood flow to the heart muscle due to insufficient blood flow to the heart muscle; and disposing an implant within the channel, wherein the implant comprises an antithrombotic or anticoagulant active agent that reduces or prevents thrombosis in the channel and/or a the implant comprises growth factor active agent that promotes angiogenesis and growth of new capillaries in the heart muscle that provide additional blood to the heart muscle which alleviates the insufficient blood flow to the heart muscle.

The second embodiments may have one or more, or any combination of the following aspects (1)-(8): (1) wherein the growth factor comprises basic fibroblast growth factor (bFGF), acidic FGF, vascular endothelial growth factor, platelet derived growth factor, stem cells, and any combination thereof; (2) wherein the implant comprises a tubular body comprising walls surrounding a lumen through which the blood flows; (3) wherein the implant comprises a tubular body comprising walls surrounding a lumen, wherein the walls are fenestrated, porous, contain pores, or have open cells; (4) wherein the implant is bioresorbable and completely resorbs away after releasing the active agent; (5) wherein the implant comprises a bioresorbable polymer; (6) wherein the implant comprises a coating including the active agent; and wherein the implant comprises a tubular body, the method further comprising radially expanding the tubular body after being disposed in the channel to an outer diameter larger than a diameter of the channel which provides for increased blood flow; (7) wherein the channel extends from a ventricle through the heart muscle or myocardium to the pericardium to allow oxygen-rich blood to flow into the channel; and (8) wherein the channel is formed from an endocardial or ventricle side of the heart and the implant is delivered percutaneously into the channel from the ventricle into the myocardium.

The third embodiments of the present invention include a method of treating insufficient blood flow to a heart muscle comprising: creating a channel in a heart muscle of a patient in need of increased blood flow to the heart muscle due to insufficient blood flow to the heart muscle; disposing a hollow elongate implant within the channel, wherein a bioresorbable structure is disposed within the hollow elongate implant and prevents blood flow through the hollow elongate implant; wherein the bioresorbable implant comprises at least one active agent that is released in the heart muscle while blood flow is prevented, wherein after a period of release of the at least one active agent, bioresorption of the structure allows blood flow through the implant.

The third embodiments may have one or more, or any combination of the following aspects (1)-(9): (1) wherein the at least one active agent comprises an effective amount of growth factor that promotes angiogenesis and growth of new capillaries in the heart muscle that provides additional blood to the heart muscle which alleviates the insufficient blood flow to the heart muscle; (2) wherein the implant comprises a tubular body comprising walls surrounding a lumen through which the blood flows; (3) wherein the implant comprises a tubular body comprising walls surrounding a lumen, wherein the walls are fenestrated, porous, contain pores, or have open cells; (4) wherein the implant is bioresorbable and completely resorbs away after releasing the active agent; (5) wherein the implant comprises a bioresorbable polymer; (6) wherein the implant comprises a coating including the active agent; (7) further comprising radially expanding the hollow elongate implant after being disposed in the channel to an outer diameter larger than a diameter of the channel which provides for increased blood flow; (8) wherein the channel extends from a ventricle through the heart muscle or myocardium to the pericardium to allow oxygen-rich blood to flow into the channel; and (9) wherein the channel is formed from an endocardial or ventricle side of the heart and the implant is delivered percutaneously into the channel from the ventricle into the myocardium.

The fourth embodiments of the present invention include an implant for treating insufficient blood flow to a heart muscle comprising: a hollow elongate body comprising walls around a lumen, wherein the hollow elongate body comprises a bioresorbable polymer, wherein upon implantation in a channel in a heart muscle the hollow elongate body supports the channel which provides increased blood flow to the heart muscle; an effective amount of an antithrombotic or anticoagulant active agent that reduces or prevents thrombotic closure of the channel; and an effective amount of a growth factor active agent that promotes angiogenesis and growth of new capillaries in the heart muscle that provides additional blood to the heart muscle which alleviates the insufficient blood flow to the heart muscle.

The fourth embodiments may have one or more, or any combination of the following aspects (1)-(6): (1) wherein the hollow elongate body is tubular and has an inside diameter of 1 to 2 mm; (2) wherein the elongate body is a radially expandable scaffold that is capable of being radially expanded at 37° C.; (3) wherein the hollow elongate body comprises a coating including a polymer and the antithrombotic or anticoagulant active agent; (4) wherein the hollow elongate body comprises a coating including a polymer and the growth factor active agent; (5) wherein the antithrombotic or anticoagulant active agent is disposed on an inner surface of the hollow elongate body and the growth factor active agent is disposed on an outer surface of the tubular body; and (6) further comprising a plurality of the implants disposed in a sealed package.

The fifth embodiments of the present invention include an implant for treating insufficient blood flow to a heart muscle comprising: a hollow elongate body comprising walls around a lumen, wherein the hollow elongate body is made of a bioresorbable polymer, and a bioresorbable sponge inside the hollow elongate body, wherein the sponge contains an effective amount of antithrombotic or anticoagulant active agent and an effective amount of growth factor active agent(s) that promotes angiogenesis and growth of new capillaries in the heart muscle that provides additional blood to the heart muscle which alleviates the insufficient blood flow to the heart muscle when and after the polymers are degraded away.

The fifth embodiments may have one or more, or any combination of the following aspects (1)-(5): (1) wherein the walls of the hollow elongate body contain multiple holes; (2) wherein the hollow elongate body is tubular and has an inside diameter of 1 to 2 mm; (3) wherein the hollow elongate body is a radially expandable scaffold that is capable of being radially expanded at 37° C.; (4) wherein the sponge is made of a hydrogel or a bioresorbable polymer; and (5) further comprising a plurality of the implants disposed in a sealed package.

The sixth embodiments of the present invention includes a bioresorbable implant for use in treatment of insufficient blood flow to a heart muscle in a patient in need thereof, wherein: the bioresorbable implant is disposed in a channel created in a heart muscle of the patient, the bioresorbable implant comprises a bioresorbable hollow elongate tubular body comprising a lumen, wherein the body includes a bioresorbable polymer, and the bioresorbable implant is capable of supporting and maintaining at least a portion of the channel to allow oxygen rich blood to flow through the channel.

The sixth embodiments may have one or more, or any combination of the following aspects (1)-(8): (1) wherein walls of the bioresorbable hollow elongate tubular body are fenestrated, porous, contain pores, or have open cells; (2) wherein the bioresorbable hollow elongate body comprises a conduit or tube that is nonporous and free of holes in the walls of the tube; (3) wherein the implant is bioresorbable and is capable of completely resorbing away from the channel after providing the support to the channel; (4) wherein the bioresorbable implant comprises an antithrombotic or anticoagulant active agent, a growth factor active agent, or both; (5) wherein the bioresorbable implant is capable of being radially expanded after being disposed in the channel to an outer diameter larger than a diameter of the channel which provides for increased blood flow; (6) wherein the channel extends from a ventricle through the heart muscle or myocardium to the pericardium to allow oxygen-rich blood to flow into the channel; and (7) wherein the channel is formed from an endocardial or ventricle side of the heart and the bioresorbable implant is delivered percutaneously into the channel from the ventricle into the myocardium; and (8) further comprising a plurality of the implants disposed in a sealed package.

The seventh embodiments of the present invention includes a bioresorbable implant for use in treatment of insufficient blood flow to a heart muscle in a patient in need thereof, wherein: the bioresorbable implant is disposed within a channel created in a heart muscle of the patient, the bioresorbable implant comprises an antithrombotic or anticoagulant active agent, a growth factor active agent, or both, and the bioresorbable implant is capable of releasing: the antithrombotic or anticoagulant active agent to reduce or prevent thrombosis in the channel and/or the growth factor active agent to promote angiogenesis and growth of new capillaries in the heart muscle to provide additional blood to the heart muscle which alleviates the insufficient blood flow to the heart muscle.

The seventh embodiments may have one or more, or any combination of the following aspects (1)-(9): (1) wherein the antithrombotic or anticoagulant active agent is selected from the group consisting of sodium heparin, low molecular weight heparin, solvent soluble heparin such as TDMAC-heparin, benzalkonium heparin, fondaparinux, idraparinus, Xa inhibitor, coumadins, hirudin and its derivatives, EDTA and any combination thereof; (2) wherein the growth factor comprises basic fibroblast growth factor (bFGF), acidic FGF, vascular endothelial growth factor, platelet derived growth factor, stem cells, and any combination thereof; (3) wherein the bioresorbable implant comprises a bioresorbable hollow elongate body surrounding a lumen and walls of the body are fenestrated, porous, contain pores, or have open cells; (4) wherein the bioresorbable implant comprises a bioresorbable hollow elongate body surrounding a lumen that is nonporous and free of holes in the walls of the bioresorbable tubular body; (5) wherein the implant is bioresorbable and is capable of completely resorbing away from the channel after releasing the active agent; and (6) wherein the bioresorbable implant is capable of being radially expanded after being disposed in the channel to an outer diameter larger than a diameter of the channel which provides for increased blood flow; (7) wherein the channel extends from a ventricle through the heart muscle or myocardium to the pericardium to allow oxygen-rich blood to flow into the channel; (8) wherein the channel is formed from an endocardial or ventricle side of the heart and the bioresorbable implant is delivered percutaneously into the channel from the ventricle into the myocardium; and (9) further comprising a plurality of the implants disposed in a sealed package.

The eighth embodiments of the present invention include a bioresorbable implant for use in treatment of insufficient blood flow to a heart muscle in a patient in need thereof, wherein: the bioresorbable implant is disposed within a channel created in a heart muscle of the patient, the bioresorbable implant comprises a bioresorbable hollow elongate body surrounding a lumen and a bioresorbable structure disposed within the lumen, the bioresorbable structure is capable of partially or completely obstructing blood flow through the lumen of the bioresorbable hollow elongate body, wherein the bioresorbable implant comprises at least one active agent and is capable of releasing the at least one active agent in the heart muscle while blood flow is prevented, and after a period of release of the at least one active agent, the bioresorbable structure is capable of resorption which reduces the obstruction to blood flow through the bioresorbable tubular implant.

The eighth embodiments may have one or more, or any combination of the following aspects (1)-(7): (1) wherein the at least one active agent comprises an effective amount of growth factor that promotes angiogenesis and growth of new capillaries in the heart muscle that provides additional blood to the heart muscle which alleviates the insufficient blood flow to the heart muscle; (2) wherein the bioresorbable structure comprises a bioresorbable sponge; (3) wherein the bioresorbable implant is capable of being radially expanded after being disposed in the channel to an outer diameter larger than a diameter of the channel which provides for increased blood flow; and (4) wherein the channel extends from a ventricle through the heart muscle or myocardium to the pericardium to allow oxygen-rich blood to flow into the channel; (5) wherein the channel is formed from an endocardial or ventricle side of the heart and the bioresorbable implant is delivered percutaneously into the channel from the ventricle into the myocardium; (6) wherein the bioresorbable structure comprises the at least one active agent; and (7) further comprising a plurality of the implants disposed in a sealed package.

The ninth embodiments of the present invention include a method of treating insufficient blood flow to a heart muscle comprising: creating a plurality of channels in a heart muscle of a patient in need of increased blood flow to the heart muscle due to insufficient blood flow to the heart muscle; disposing a plurality of implants within the channels, wherein the implants support and maintain at least a portion of the channels to allow oxygen rich blood to flow through the channels.

The ninth embodiments may have one or more, or any combination of the following aspects (1)-(8): (1) wherein each of the implants comprise a shape defined by a wall that encloses a cavity or lumen; (2) wherein the implants comprise an hollow elongate body comprising walls surrounding a lumen through which the blood flows; (3) wherein the implants comprise hollow elongate bodies comprising walls surrounding lumens, wherein the walls of the bodies are fenestrated, porous, contain pores, or have open cells; (4) wherein the implants are bioresorbable and completely resorb away from the channels after providing the support to the portion of the channel; (5) wherein the implants comprise bioresorbable polymer; and (6) wherein the implants comprise hollow elongate bodies, the method further comprising radially expanding the bodies after being disposed in the channels to an outer diameter larger than a diameter of the channel which provides for increased blood flow; (7) wherein the channels extend from a ventricle through the heart muscle or myocardium to the pericardium to allow oxygen-rich blood to flow into the channels; and (8) wherein the channels are formed from an endocardial or ventricle side of the heart and the implant is delivered percutaneously into the channels from the ventricle into the myocardium.

### BRIEF DESCRIPTION OF THE DRAWINGS

FIG. 1 depicts a schematic illustration of a transmyocardial revascularization procedure.

FIG. 2 depicts a section of a tube.

FIG. 3 depicts an exemplary scaffold.

### DETAILED DESCRIPTION OF THE INVENTION

Embodiments of the present invention include an implant for treating insufficient blood flow to a heart muscle with transmyocardial revascularization (TMR). Embodiments also include methods of treating insufficient blood flow to a heart muscle with the implant using transmyocardial revascularization (TMR).

There are at least two critical aspects to TMR. First, channels that are created in the heart muscle act as bloodlines so that when the ventricle pumps or squeezes oxygen-rich blood out of the heart, it sends blood through the channels which increases perfusion of the heart muscle. In some embodiments, holes will be drilled to place the implant. The holes may be 1.5 to 3 mm in diameter. The hole(s) may be sealed from the pericardial side since the blood from ventricle may shoot out if not sealed.

Second, the increased blood flow provided by the channels, combined with the released growth factor, promotes angiogenesis or growth of new capillaries (small blood vessels) that help supply blood to the heart muscle. It is also important that thrombosis does not develop in the channels and restrict blood flow through the channels.

In TMR, the channels created may decrease in size with time and eventually seal up. Thus, the increased blood flow through the channels may decrease with time and eventually may cease completely. The growth of new capillaries due to the increased blood flow from the channels is believed to provide long-term increased blood flow to the heart muscle which alleviates the angina caused by insufficient blood flow to the heart muscle.

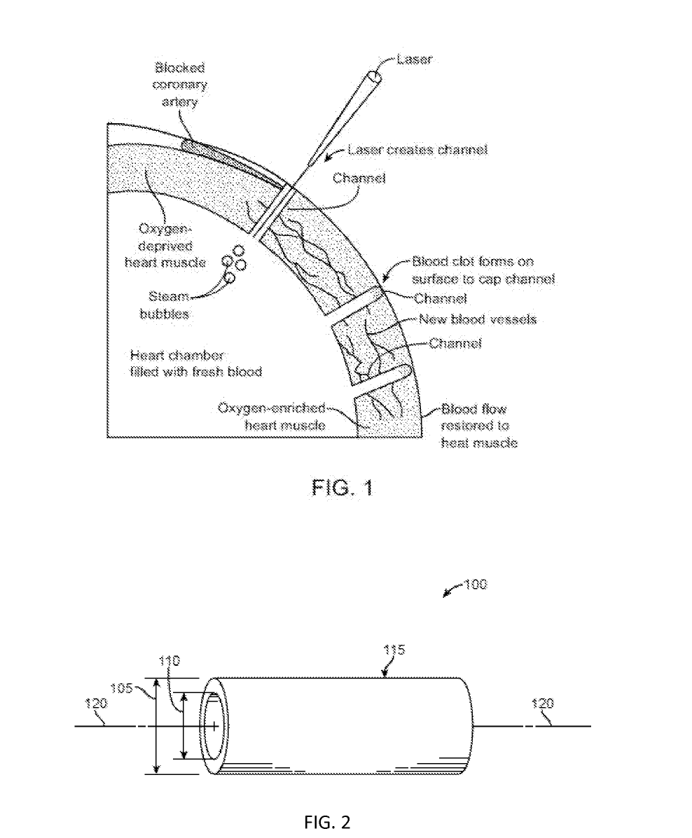

FIG. 1 depicts a schematic illustration of a TMR procedure. FIG. 1 shows a section of a human heart that includes a heart muscle or myocardium enclosing a chamber or ventricle of the heart which is filled with fresh blood. As shown, a blocked coronary artery results in oxygen-deprived heart muscle. A laser is used to create channels in the heart muscle from the pericardial side of the heart muscle which extend through the heart muscle to the ventricle. As shown, the process results in steam bubbles that form in the ventricle near the opening of the channel. Fresh blood flows from the ventricle through the channels which restores flood flow to the oxygen-deprived heart muscle. New blood vessels are formed through angiogenesis due to the fresh blood flow which facilitates oxygen enrichment of heart muscle. As shown, eventually a blood clot forms at the surface of the channel opening on the pericardial side to cap the channel.

Embodiments of the present invention address these aspects of TMR to maintain and promote increased blood flow to a heart muscle treated with TMR.

Certain embodiments of the invention include creating at least one channel in a heart muscle or myocardium of a patient in need of increasing blood flow to the heart muscle due to insufficient blood flow to the heart muscle. The insufficient blood flow to the heart muscle may be due to a stenotic artery near the heart muscle that is blocked or partially blocked. The channels may extend from a ventricle through the myocardium to the pericardium to allow oxygen-rich blood to flow into the channels from the ventricle. The channel may be creating an opening in pericardium and forming the channel through the myocardium. Alternatively, the channel may be formed, as described herein, at the endocardial side from the ventricle. In this embodiment, the channel may extend all the way through the myocardium and the pericardium with an opening on the pericardial side. In another aspect, the channel may extend partially through the myocardium.

The number of channels created can be 10, 2 to 10, 2 to 5, 2 to 50, 4 to 40, 3 to 30, or 5 to 20. The channels may have a circular cross-section and have a diameter of 0.5 to 1 mm, 1 to 1.5 mm, 1.5 to 3 mm, or greater than 3 mm.

The present invention further includes disposing an implant with an inner lumen within the channel. The lumen provides a flow path for blood flowing into and through the channel. The implant may be a conduit, tubular, or elongate, and the implant may be disposed so that its longitudinal axis coincides with the longitudinal axis of the channel. Disposing the implant may include inserting the implant within the channel so that an outer surface of the implant is in apposition to and in contact with a wall or surface that defines the channel. The implant may maintain at least a portion of the channel to allow oxygen rich blood from the ventricle to flow through the channel. The implant supports the walls of the channel and reduces or prevents the decrease in size of the channel. Therefore, the implant maintains the opening or flow path for blood through the channel for a longer time. As a result, the perfusion in the region of ischemic myocardium is increased due to the implant. In additional or alternative embodiments, the implant may include active agents or drug and delivers the drugs to prevent or inhibit thrombotic closure of the channel, to promote vascularization, or both. Specifically, an antithrombotic agent may be released from the implant that prevents or reduces thrombosis in the channel. Additionally or alternatively, the implant can include an active agent that is a growth factor that promotes angiogenesis or growth of new capillaries that help supply blood to the heart muscle.

The blood flow through the channels may be necessary for a limited or finite time. After a certain period of time, the increased blood flow from the channels may promote new capillary growth that is sufficient to restore blood flow to the heart muscle. Therefore, the support provided by the implant to the channels may be necessary for a limited time period. As a result, the presence of the implant may be required for a limited time.

In an alternative embodiment, the inner lumen of the implant is partially or completely obstructed or blocked with a bioresorbable, biosoluble structure or plug made of a material this is bioresorbable, biosoluble, or a combination thereof such as a hydrogel. Bioresorbable and biosoluble may be used synonymously. In this approach, blood flow through the implant inner lumen may be partially or completely obstructed, blocked, or restricted for period of time after implantation. For example, a porous structure, such as a sponge, made of the bioresorbable or biosoluble material may be embedded in the inner lumen of the implant. As the bioresorbable or biosoluble material is resorbed, dissolved, etc., the implant inner lumen becomes unblocked and blood will low through the implant inner lumen or become less blocked and blood flow will increase through the implant inner lumen. Additionally, the bioresorbable or soluble material may include an active agent that can be released into the heart during the time that the artery is blocked or partially blocked.

The blood flow through the channels may not be necessary initially for a limited time to allow the active or biological agent embedded in the sponge to diffuse to the surrounding tissue through openings in the implant. Here, sponge is defined as any bioresorbable or elutable materials that host bioactive agent(s). After a certain period of time, sponge is resorbed or dissolved and the blood flow is increased in the channels, which may promote new capillary growth that is sufficient to restore blood flow to the heart muscle. Therefore, the support provided by the implant to the channels may be necessary for a limited time period, but is longer than the sponge resorption or elution time. As a result, the presence of the implant may be required for a limited time.

Thus, the implant may be made partially or completely out of a bioresorbable material. After the implant has served its function of increasing blood flow that promotes new capillary growth which provides increased perfusion of the heart muscle, the implant may partially or completely disappear from the treatment location by resorbing. The implant performs this function by providing mechanical support or patency to the channel, provides drug delivery to enhance angiogenesis, or both. Embodiments can include implants fabricated from biodegradable, bioabsorbable, bioresorbable, biosoluble and/or bioerodable materials such as bioabsorbable polymers or bioerodible metals that can be designed to completely erode only after the clinical need for them has ended.

The bioresorbable material for the implant may be bioresorbable polymer. Exemplary bioresorbable polymers for implant include polylactide (PLA)-based polymers, polycaprolactone, poly(glycolide), polydioxanone, polytrimethylene carbonate, and poly(4-hydroxybutyrate), poly(3-hydroxybutyrate), or a copolymer or blend of any combination of the above polymers. PLA-based polymers include poly(L-lactide), poly(D-lactide), poly(D,L-lactide), poly(D,L-lactide) having a constitutional unit weight-to-weight (wt/wt) ratio of about 96/4, poly(L-lactide-co-D,L-lactide), poly(L-lactide-co-glycolide), poly(D,L-lactide-co-glycolide), poly(L-lactide-co-caprolactone), poly(D,L-lactide-co-caprolactone), poly(D,L-lactide) made from meso-lactide, and poly(D,L-lactide) made from polymerization of a racemic mixture of L- and D-lactides. A PLA-based polymer can include a PLA with a D-lactide content greater than 0 mol % and less than 15 mol %, or more narrowly, 1 to 15 mol %, 1 to 5 mol %, 5 to 10%, or 10 to 15 mol %. The PLA-based polymers include poly(D,L-lactide) having a constitutional unit weight-to-weight (wt/wt) ratio of about 93/7, about 94/6, about 95/5, about 96/4, about 97/3, about 98/2, or about 99/1. The term “unit” or “constitutional unit” refers to the composition of a monomer as it appears in a polymer

The number average molecular weight (Mn) of the polymer implant material may be 50 to 100 kDa, 50 to 60 kDa, 60 to 80 kDa, 80 to 100 kDa, greater than 50 kDa, or greater than 100 kDa.

Exemplary bioresorbable material for the sponge or bioresorbable structure includes any of the above bioresorbable polymers. The sponge or bioresorbable structure may also be made of a hydrogel which is a crosslinked hydrophilic polymer that can absorb a large amount of water. Exemplary hydrogels can be made from polyethylene glycol (PEG), hyaluronic acid (HA), or HA-poly(ethylene oxide) (PEO), or poly(vinyl alcohol). The hydrogels can also be crosslinked block copolymers of hydrophilic polymers and bioresorbable polymers, such as any of those disclosed herein above.

Any combination of the polymers for the implant material and plug or sponge may be used. The plug or sponge may be made of a polymer that degrades or dissolves faster than the implant material degrades. The implant, plug, or sponge may be made partially or completely or any of the polymers disclosed herein or any combination of the polymers disclosed herein. The implant may provide support which allows increased blood flow to the channel for 2 to 24 months, 2 to 12 months, 2 to 4 months, 2 to 6 months, 4 to 6 months, or 4 to 12 months. The implant may completely resorb from the channel in 2 to 30 months, 3 to 12 months, 3 to 24 months, 3 to 12 months, 6 to 30 months, or 6 to 12 months.

The implant structure includes a body defined by a wall that encloses a cavity or lumen. When the implant is disposed in the channel, the walls support and maintain the size of the at least a portion of the channel. The lumen or cavity is the interior of the supported portion of the channel. The supported portion of the cavity provides for increased blood flow through the lumen or cavity.

In some embodiments, the implant is an elongate structure such as a hollow elongate body with a wall surrounding an inner lumen. The lumen may have a circular transverse cross-section, or generally, other shapes such as square, rectangular, oval, etc. In particular, the implant may be a conduit or tubular construct. The walls of the tubular construct enclose a lumen through which blood flows when the tubular implant is implanted. The tubular implant is disposed into the channel with the outer surface of the tube in apposition to and in contact with the walls or tissue that define the channel. The inner surface of the channel is in contact with the lumen of the channel.

The walls of the implant structure can have gaps or holes that extend between the inner and outer surface of a wall so that the tissue of the walls of the channel is exposed to the lumen through the gaps or holes. Alternatively, the walls of the structure can be free of such gaps or holes. Additionally, the walls of the structure can be porous with closed or open cell pores throughout the wall material or in a portion of the wall material.

The inside diameter of the tubular implants may be 1 mm, 0.05 to 1.05 mm, 1 to 2 mm, 1.2 to 2 mm, 1.4 to 2 mm, 2 to 2.5 mm, 2.5 to 3 mm, or greater than 3 mm. In order to account for the wall thickness of the implant, the channel size or diameter may be larger than that used in conventional TMR. For example, the diameter of channels may be at least the diameter of the channels in conventional TMR plus twice the wall thickness of the tubular implant. The outside diameter of the channels may equal to the diameter of the channel as formed without the implant. The outside diameter of the implant may be equal to the diameter of the channel. The outside diameter of the implant may be 1 to 1.1 times, 1.1 to 1.2, or 1.2 to 1.3 times the diameter of the channel as formed without the implant.

The implantation can be achieved through pericardial cut down. After making an incision in the pericardium, the channels can be created or formed using mechanical, chemical, thermal, or optical techniques. Mechanical techniques include hole puncturing and an optical technique includes lasing drilling. The implant may be inserted by using a punch-like delivery device.

Alternatively, the channel can be formed from the endocardial or ventricle side of the heart and the implant may be delivered percutaneously using a catheter device similar to Mitraclip of Abbott Laboratories and implanted from the ventricle into the myocardium. After implantation, the epicardial opening can be sealed by a suture or a vessel closure device. In such a procedure, the implant may be attached to the catheter which may be a steerable guide catheter. The catheter is advanced within the guide through the body of the patient guide to the endocardial or ventricle side of the heart. The implant may be attached, compressed, or crimped onto a catheter and then deployed once it is inserted into a channel in the myocardium. The deployment may include expanding the implant within the channel by expanding a balloon catheter.

The implant may be delivered with the size or dimension in which it is intended to function upon implantation. Therefore, the implant may be fabricated and implanted with its as-fabricated dimensions. For example, a tubular implant may be fabricated having a specified diameter and then implanted in a channel with this diameter.

In such embodiments, the tubular implant is not capable of self expansion in its as-fabricated or as-delivered configuration. The tubular implant may also not be balloon expandable in its as-delivered or as-fabricated configuration. The implant may be delivered mounted over a support that cannot radially expand the implant.

Alternatively, the implant may be delivered having a dimension smaller than an as-fabricated condition. A tubular implant may be delivered by first crimping the implant from an as-fabricated diameter to a reduced diameter. Upon insertion into the channel, the implant may be expanded from a reduced diameter to a target diameter. The outer target diameter may be the same as the desired inner diameter of the channel.

The implant may also be expanded to an outer diameter larger than the channel diameter. The expanded diameter of the channel provides for an even greater increased blood flow. For example, the channel diameter may be the diameter for conventional TMR and the implant may be expanded to account for the wall thickness of the tubular implant.

The channels in the heart muscle may be created by a laser, for example a CO2 laser, in particular, the CO2 Heart Laser 2 that may be obtained from PLC Medical Systems of Milford, Mass. A computer is used to direct laser beams to the appropriate area of the heart in between heartbeats, when the ventricle is filled with blood and the heart is relatively still.

An effective amount of active agents or drugs to prevent thrombotic closure of the implant or to promote vascularization can be included or incorporated in the implant in various ways. The drugs can be incorporated into the implant structure, for example, within the walls of the implant. The drug may be distributed throughout the wall of the implant. Alternatively or additionally, the implant may include a coating over the implant that includes the drug. The coating may include a polymer carrier with the drug distributed within the polymer. Alternatively or additionally, the implant may have an inner layer and an outer layer with one of the layers including one drug and another layer including another drug or no drug.

The walls of an implant require sufficient strength to maintain its shape and dimensions to support the channel. Specifically, the walls must be able to resist the compressive forces of the beating heart. In the case of a tubular structure, the implant requires radial strength sufficient to resist the radial compressive forces of the beating heart to maintain its shape and thus the channel size. In particular, the implant must be able to resist the systolic/diastolic pressure of the beating heart. During each heartbeat, blood pressure varies between a maximum (systolic) and a minimum (diastolic) pressure. As shown in Tables 1 and 2, the magnitude of these pressures depends on several factors such as age of the patient and the existence and degree of hypertension in the patient.

TABLE 1
Classification of blood pressure for adults. [1], [2]

| Category | systolic, mmHg | diastolic, mmHg |
|---|---|---|
| Hypotension | <90 | <60 |
| Desired | 90-119 | 60-79 |
| Prehypertension | 120-139 | 80-89 |
| Stage 1 Hypertension | 140-159 | 90-99 |
| Stage 2 Hypertension | 160-179 | 100-109 |
| Hypertensive Crisis | ≧180 | ≧110 |

[1] “Understanding blood pressure readings”. American Heart Association. 11 Jan. 2011. Retrieved 30 Mar. 2011.
[2] “Low blood pressure (hypotension) - Causes”. MayoClinic.com. Mayo Foundation for Medical Education and Research. 2009 May 23. Retrieved 2010 Oct. 19.

TABLE 2
Reference ranges of blood pressure.

| Stage | Approximate age | | Systolic | Diastolic |
|---|---|---|---|---|
| Infants | 1 to 12 | months | 75-100^{[19]} | 50-70^{[19]} |
| Toddlers | 1 to 4 | years | 80-110^{[19]} | 50-80^{[19]} |
| Preschoolers | 3 to 5 | years | 80-110^{[19]} | 50-80^{[19]} |
| School age | 6 to 13 | years | 85-120^{[19]} | 50-80^{[19]} |
| Adolescents | 13 to 18 | years | 95-140^{[19]} | 60-90^{[19]} |

^{[19]}PEDIATRIC AGE SPECIFIC, page 6. Revised June 2010. By Theresa Kirkpatrick and Kateri Tobias. UCLA Health System.

Radial strength, which is the ability of a tubular implant to resist radial compressive forces, relates to an implant's radial yield strength and radial stiffness around a circumferential direction of the implant. An implant's “radial yield strength” or “radial strength” (for purposes of this application) may be understood as the compressive loading or pressure, which if exceeded, creates a yield stress condition resulting in the implant diameter not returning to its unloaded diameter, i.e., there is irrecoverable deformation of the implant in the radial direction. See, T. W. Duerig et al., Min Invas Ther & Allied Technol 2000: 9(3/4) 235-246.

Radial stiffness is a measure of the elastic response of an implant to an applied load and thus will reflect the effectiveness of the implant in resisting diameter loss due to lumen (in this case channel) recoil and other mechanical events. Radial stiffness can be defined for a tubular implant as the hoop force per unit length (of the implant) required to change its diameter through elastic deformation. Thus, even when an implant has a high radial strength and can resist irrecoverable radial deformation, a low radial stiffness results in higher deviations in the diameter of the implant as the pressure exerted on the implant varies. The inverse or reciprocal of radial stiffness may be referred to as the radial compliance. See, T. W. Duerig et al., Min Invas Ther & Allied Technol 2000: 9(3/4) 235-246.

Once disposed within the channel, the implant must adequately provide channel support during a time required for treatment in spite of the various forces that may come to bear on it, including the cyclic loading induced by the beating heart. The radial strength of an implant depends on material properties, material processing and geometric or dimensional properties of the implant. The time required for treatment may correspond to the time for a sufficient new capillary growth that restores blood flow to the heart muscle and alleviates symptoms of reduced blood flow, such as angina.

Geometric or dimensional properties include the thickness of the walls of the implant and the macroscopic structure of the wall, such as holes or gaps in the wall and the porosity of the wall. Gaps or holes in the wall or porosity may be desirable to promote tissue ingrowth around the implant, however, they can decrease the radial strength and stiffness of the implant.

Material properties include mechanical properties such as the strength and tensile modulus of the implant material. The higher the strength of the implant material, the higher the radial strength of the implant is expected to be. In addition, the higher the stiffness of the implant material, the higher is the radial stiffness.

Additionally, the radial strength of the implant also depends on the crystallinity of and polymer chain orientation in polymeric implant material. The strength of the material and the radial strength of the implant depend on the degree of crystallinity of the polymer. Increasing the degree of crystallinity increases the strength and stiffness of the material and the radial strength and stiffness of the implant. Also, the preferential orientation of polymer chains also influences the radial strength and stiffness of the implant. A preferential orientation in the circumferential direction increases radial strength and stiffness. Varying degrees of crystallinity and radial orientation may be achieved through the processing used to make the implant.

The radial strength of the implant may be greater than 200 mm Hg, 200-300 mm Hg, 300 to 400 mm Hg, 300 to 600 mm Hg, higher than 400 mm Hg, or higher than 600 mm Hg.

The implant may be made partially or completely of a high strength, high modulus polymer that provides high radial strength and stiffness to the implant under physiological conditions. Such polymers may be semicrystalline and include crystalline regions in an amorphous polymer matrix. The degree of crystallinity of semicrystalline polymers can vary and depends on the processing history and the chemical composition of the polymer. The degree of crystallinity can be 10 to 75%, or more narrowly, 10 to 30%, 30 to 50%, or 50 to 70%, or greater than 70%.

The implant may be totally amorphous, i.e., less than 5% crystallinity or 0% crystallinity. Through a manufacturing process, partial oriented amorphous morphology could be formed to enhance the radial strength of the tubular implant.

Table 3 compares the properties of several bioresorbable polyesters. As shown in the table, poly(L-lactide) (PLLA) and polyglycolide (PGA) have a relatively high strength and high modulus. The high strength and high modulus polymer for use in as an implant material can include copolymers and blends PLLA and PGA, for example, poly(L-lactide-co-glycolide) (PLGA). The PLGA can have a mole % of GA between 5 and 50 mol %, or more narrowly, 5-15 mol %. The PLGA can have a mole % of (LA:GA) of 85:15 (or a range of 82:18 to 88:12), 50:50 (or a range of 48:52 to 52:48), 95:5 (or a range of 93:7 to 97:3), or commercially available PLGA products identified being 85:15, 50:50, or 95:5 PLGA.

TABLE 3
Comparison of properties of bioabsorbable polymers. Martin
et al., Biochemical Engineering 16 (2003) 97-105.

| | | | Tensile | Tensile | | |
|---|---|---|---|---|---|---|
| | Tg | Tm | Strength | Modulus | Absorption | |
| | (° C.) | (° C.) | (MPa) | (MPa) | Time | |
| PLLA | 175 | 65 | 28-50 | 1200-2700 | 1.5-5 | years |
| P4HB* | 60 | −51 | 50 | 70 | 8-52 | weeks |
| PCL* | 57 | −62 | 16 | 400 | 2 | years |
| PGA | 225 | 35 | 70 | 6900 | 6 | months |
| PDLLA* | Amorphous | 50-53 | 16 | 400 | 2 | years |
| P3HB* | 180 | 1 | 36 | 2500 | 2 | years |

*P4HB—poly(4-hydroxybutyrate); PCL—polycaprolactone; PDLLA—poly(DL-lactide); P3HB—poly(3-hydroxybutyrate).

The high strength and high modulus polymer for use as an implant material may be characterized by several properties and may have one or any combination of such properties. The properties may correspond to the polymer prior to processing into an implant or the property of the polymer as part of a fabricated implant. The polymer may have a tensile strength greater than 10 MPa, 20 MPa, 30 MPa, 40 MPa, 50 MPa, 60 MPa, or 70 MPa or between 10 and 20 MPa, 20 and 30 MPa, 30 and 50 MPa, or 50 and 70 MPa. The polymer may have an elongation at break less than 20%, 10%, 5%, or 3% or between 3 and 5%, 5 and 10%, or 10 and 20%. The polymer may have a modulus of elasticity greater than 0.2 GPa, 1 GPa, 2 GPa, 3 GPa, 5 GPa, or 7 GPa or between 0.2 and 2 GPa, 2 and 3 GPa, 3 and 5 GPa, or 5 and 7 GPa. The properties may correspond to a wet or dry state at 25° C. or 37° C. The wet state may correspond to a soaking of the material in a water, phosphate buffered saline solution, blood, or in simulated body fluid. The soaking time can at least 2 minutes or until the material is saturated.

Additionally, the polymer may have a glass transition temperature (Tg) greater than body temperature or 37° C., or greater than 10° C. or greater than 20° C. above human body temperature or 37° C. The polymer may have one or any combination of such properties. The property or properties may refer to a copolymer or blend of polymers.

Other high strength, high modulus polymers for use as an implant material include tyrosine carbonate copolymers, polydioxanone, polyacetylsalicylic acid, copolymers of PLLA and another bioresorbable polymer, copolymers of PGA and another bioresorbable polymer.

In other embodiments, the implant may be made partially or completely of a bioerodible metal such as zinc, iron, magnesium or an iron-based alloy or a magnesium-based alloy.

Various embodiments of the structure of an implant may be used. A tubular implant may include a tube with no gaps or holes in the wall between an inner and out surface. FIG. 2 depicts a section of a tube 100 with a wall 115 with an inside diameter 110 and an outside diameter 105 and a cylindrical or longitudinal axis 120. The wall thickness is the difference between the outside diameter 105 and inside diameter 110. The wall 115 surrounds an inner lumen. Such a tube can be formed, for example, by extrusion, dipping, or injection molding.

In other embodiments, the tubular implant can include gaps or holes in the wall between the inner and out surface. These embodiments can include a tube with a pattern of holes distributed along the surface of the wall. The size and number of the holes can be selected so that that the tubular implant has a desired radial strength and stiffness. The gaps or holes can be formed by laser cutting.

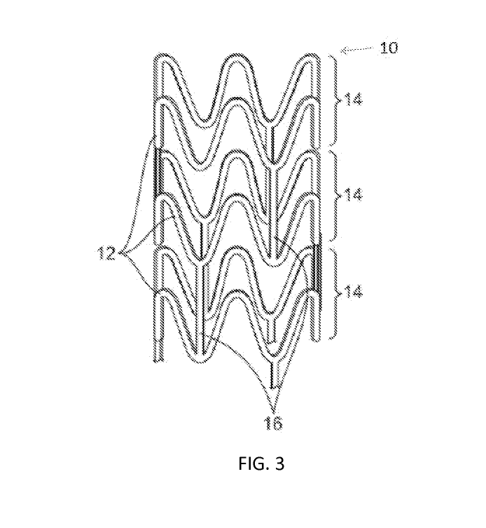

In further embodiments, the tubular implant can have a stent or scaffold structure. A scaffold may include a pattern or network of interconnecting structural elements or struts. An exemplary structure of a scaffold is shown in FIG. 3. FIG. 3 depicts a scaffold 10 which is made up of struts 12 with gaps between the struts. Scaffold 10 has interconnected cylindrical rings 14 connected by linking struts or links 16. The outer surface of the struts is the abluminal surface and the inner surface of the struts is the luminal surface. Scaffold 10 may be formed from a tube (not shown). The structural pattern of the device can be of virtually any design. The embodiments disclosed herein are not limited to scaffolds or to the scaffold pattern illustrated in FIG. 3. The embodiments are easily applicable to other patterns and other devices. The variations in the structure of patterns are virtually unlimited.

A scaffold such as scaffold 10 may be fabricated from a polymeric tube or a sheet by rolling and bonding the sheet to form the tube. The scaffold pattern can then be formed with laser cutting.

In other embodiments, a tubular implant can have porous walls that include a three dimensional network of interconnected pores. Any of the disclosed structures can have porous walls. The porous structure can be open or closed cell. The pore size (e.g., diameter) of any pores or the average pore size may be less 1 μm, 1-10 μm, 10-100 μm, or greater than 100 μm. A porous polymer tube may be formed, for example, by extrusion with supercritical carbon dioxide.

In additional embodiments, any of the disclosed embodiments of the tubular structure can be sealed at one end. The closed end may be the distal end, i.e., the open end of the implant will be inserted first into the heart muscle when inserted from the pericardial side. The closed end may be the proximal end, i.e., the closed end of the implant will be inserted first into the heart muscle when inserted from the endocardial side. The sealed tube may be fabricated, for example, by laser welding an end of a tube or scaffold.

The implants may be supplied as on or a plurality of implants disposed in a sealed package. The implants may be positioned on a delivery system in the package. The implants may be sterilized in the package.

In another embodiment, the implant can be an epicardial side sealed cylinder.

In another embodiment, the implant can be a hollow cone with the mouth facing the ventricle when inserted into the heart muscle.

In additional embodiments, the implant can have an arbitrary shape defined by a wall surrounding an inner enclosure. For example, the structure may be spherical or oblong. The walls may have holes or gaps to allow blood to flow through the inner enclosure when the implant is disposed in a channel. The spherical or oblong structure may be formed as coil balls or have a buckyball structure.

The radial strength and stiffness of an implant may be adjusted through variation of the thickness of the walls of an implant. The thickness of the walls required for a given radial strength will depend on the geometry of the implant (e.g., scaffold pattern, holes and gaps, porosity) and the strength and stiffness of the material of the implant. The thickness of the walls may be 50 to 100 microns, 100 to 150 microns, 150 to 160 microns, 160 to 200 microns, 200 to 250 microns, 250 to 300 microns, 300 to 350 microns, 350 to 400 microns, or greater than 400 microns.

The radial strength and stiffness of the implant can also be adjusted through various processing steps. The radial strength of an implant made from a polymer can be increased by annealing, deformation, or both. Both of these processing steps can increase the crystallinity of the polymer which increases the strength and stiffness of the polymer and thus increases the radial strength.

The annealing step can be performed on a polymer construct such as a tube prior to forming holes, gaps, or a scaffold from the construct. The annealing can be performed before, after, or before and after forming holes, gaps, or a scaffold from the construct.

Annealing refers to heating the construct or implant to a temperature for period of time Annealing may be performed to increase the crystallinity of the construct or implant. The annealing temperature may be at or greater than the Tg of the polymer and less than the melting point (Tm) of the polymer. The annealing temperature may be Tg to 10° C. above Tg, 10 to 20° C. above Tg, 20 to 30° C. above Tg, 30 to 40° C. above Tg, 40 to 50° C. above Tg, or greater than 50° C. above Tg. The time that the material is above Tg or in any particular temperature range above Tg may be 1 to 5 min, 5 min to 30 min, 30 min to 1 hr, 1 hr to 10 hr, 10 hr to 1 day, 1 day to 2 days, or greater than 2 days.

The annealing process can also include cooling or allowing the annealed construct to cool below the annealing temperature. The construct may be cooled or be allowed to cool to ambient or room temperature, which may be any temperature between and including 20 to 30° C. The annealed construct may be cooled by exposing it to a selected cooling temperature, such as room temperature, which can be any temperature between 20 and 30° C., or a temperature below room temperature, such as below 25 or 30° C. The annealed construct can be cooled by blowing cooled gas on the construct, disposing the construct in a refrigerator or freezer, or immersing the construct in a liquid, such as water. The annealed construct may also be quenched from the annealing temperature to a lower temperature. Quenching the construct refers to an extremely rapid cooling or extremely rapid reduction of the temperature of the polymer construct from the annealing temperature to a lower temperature such as room temperature or below room temperature, for example, 10 to 30° C. below room temperature. Quenching can be performed by exposing a polymer construct to cold liquid or gas at the above quenching temperatures ranges.

Deformation also can increase the strength and modulus of a material. The increase may be due both to an increase in crystallinity induced by the deformation, but also due to preferential polymer chain and crystallite orientation along the direction of deformation. Deforming a polymer induces a preferred orientation along the axis of deformation of the deformed polymer which increases the strength and modulus along this axis. A polymer tube prior to forming holes or scaffold may be radially expanded which induces preferred polymer chain and crystallite orientation around the circumference of the tube which increases the radial strength of the tube and an implant fabricated from the tube. Biaxial orientation can also be induced by deforming the tube along its cylindrical axis.

The percent Radial Expansion (% RE) can be defined as (IDex/IDorig−1)×100%, where IDex is the inside diameter of an expanded tube and IDorig is the original inside diameter of the tube prior to expansion. The ranges of the IDexp may correspond to the values of the desired diameters of the implants disclosed herein. The % RE may be 20% to 50%, 50% to 100%, 100% to 150%, 150% to 200%, 200% to 300%, 300% to 400%, or greater than 400%.

The tube may be radially expanded by increasing the temperature to, at, or above the Tg of the polymer(s) of the tube and increasing the pressure in the tube. The range of expansion temperatures may correspond to the annealing temperature ranges. The tube may be disposed in a tubular mold during the expansion process. The outside surface of the tube expands against the inner surface of the mold.

The annealing, deformation, or both can increase the crystallinity by 5% to 10%, 10% to 20%, 20% to 100%, 100% to 1000%, or by greater than 1000% of the original crystallinity. The crystallinity can be increased from less than 10% to 10 to 20%, 20 to 30%, 30 to 40%, 40 to 50%, 50 to 60%, or greater than 60%. Increasing the crystallinity will make the polymer brittle or more brittle, i.e., the polymer with the increased crystallinity may have a relatively low strain to fracture, e.g., less than 5%. However, for implants that are not crimped or reduced in size prior to delivery and then are expanded when implanted, the brittleness is not necessarily a disadvantage to the function of the implant. In the TMR implantation process, the implant material may not or does not undergo any or significant strain (e.g., less than 2%). As a result, the annealing or deformation temperature can be such that there is fast crystal growth resulting in large crystals, for example, larger than 20 microns or 50 microns.

The strength and stiffness of the implant material and thus the radial strength can alternatively or additionally be increased by incorporating reinforcement fillers. The filler may include particles that are distributed throughout the principal component of the implant, such as a polymer, which is a matrix. The particle material may have a strength and stiffness much higher than the matrix. The reinforcement fillers may include micro-crystalline cellulose, bioglass, hydroxyapatite, calcium phosphate, zinc, iron, magnesium and ferric oxide. The size of such particles may be less than 100 nm, 100 nm to 1 micron, 1 to 2 microns, 2 to 10 microns, or greater than 10 microns. The reinforcement fillers may be less than 1 wt %, 0.1 to 1 wt %, 1 to 5 wt %, 5 to 10 wt %, 10 to 20 wt %, or greater than 20 wt % of the implant or relative to the matrix material of the implant. Preferably, the reinforcing filler is bioresorbable or biodegradable.

To reduce or prevent thrombotic closure of the implant, the implant can include antithrombotic agents, anticoagulants, or both. Such agents can include, but are not limited to, sodium heparin, low molecular weight heparin, solvent soluble heparin such as TDMAC-heparin, benzalkonium heparin, fondaparinux, idraparinus, Xa inhibitor, coumadins, hirudin and its derivatives, EDTA and any combination thereof.

The implant can include angiogenesis promoters to promote the growth of new capillaries. Active agents that are angiogenesis promoters include, but are not limited to basic fibroblast growth factor (bFGF), acidic FGF, vascular endothelial growth factor, CD34, platelet derived growth factor, and stem cells.

The active agents can be incorporated into a carrier polymer which can include, but are not limited to, polylactide-based polymers such as poly(D,L-lactide) and copolymers thereof, polyglycolide-based polymers such as polyglycolide and copolymers thereof. Carrier polymers can also include other polyesters such as polycaprolactone, polyanhydrides such as poly(sebacic anhydride), polyhydroxyalkanoates such as poly(3-hydroxybutyrate), polyester-amide, hydrophilic polymers such as polyethylene glycol/oxide, and polyvinylpyrrolidone. Carrier polymers also include blends of the disclosed polymers and copolymers of the disclosed polymers. Additional carrier polymers include hydrogels made from polyethylene glycol, polyvinypyrolidone, polysaccharide, sugar, or copolymers thereof with a biodegradable polymer such as PDLLA, PGA, or another family of the carrier polymer.

The carrier polymer facilitates or provides controlled release of the active agents. The active agents may be released over a period of 1 day to 2 weeks, 2 weeks to 1 month, 1 to 2 months, 2 to 5 months, up to 2 months, up to 3 months, up to 5 months, or greater than 5 months.

The implant may include a base substrate or structure such as a tube or scaffold, as described herein. The active agents may be incorporated with the implant substrate in various ways. An active agent or agents may be distributed within a part or throughout the implant material of the implant substrate. An active agent coating may be disposed over an entire surface of the implant substrate or over a portion of the surface of the implant substrate. A coating with a particular agent or agents may be disposed exclusively over an inside surface, outside surface, or both.

An implant may be a tube or formed from a tube (e.g., in the case of a scaffold) having two layers, an inside layer and outside layer. The two layer tube may be formed from an inner tube and an outer tube. The inner tube and outer tube may be prepared separately and assembled to form a coaxial configuration in which the outside surface of the inner tube is attached to the inside surface of the outer tube. Alternatively, the two layer tube can be formed by coextruding layers of two types of polymers. A scaffold implant can be fabricated by laser cutting the two layer tube. One or both of the layers can be porous.

The inner tube layer may be made from a high strength, high modulus bioresorbable polymer, as described above, to provide mechanical support. The inner tube layer may be annealed or radially expanded to increase strength. The inner tube layer may be a magnesium-based bioerodable metal to provide strong mechanical support. The outer tube may be made from lower modulus bioresorbable polymers or a mixture thereof and include active agents for controlled release of active agents to prevent thrombotic closure and to promote vascularization.

Active agent(s) can be applied directly to the implant without a carrier polymer, or mixed with a carrier polymer and then applied to the scaffold. For example, TDMAC-heparin can be applied over a bFGF coated implant.

Active agent(s) can be applied directionally. A coating including an antithrombotic drug may be applied only to an inner surface of an implant and a coating containing a growth factor active agent may be applied only to an outer surface of the implant. For example, heparin coating is applied only on an inside surface of the scaffold and a coating with growth factor is applied only on the outside of the scaffold. Similarly, in a two layer implant, only the inner layer can includes the antithrombotic agent and only the outer layer can include the growth factor active agent.

Alternatively, the active agent(s), such as the growth factor can be applied between the scaffold backbone and a polymer coating, which may contain a fast eluting active agent such as heparin.

Alternatively, the active agents such as heparin or its derivative can be incorporated into the tubular implant through extrusion.

Alternatively, one active agent can be incorporated into the tubular implant and the other active agent such as bFGF can be incorporated into the hydrogel or sponge that is inserted into the tubular implant.

Application of a coating can be through dip-coating, spray-coating, or roller-coating.

Alternatively, active biological agents can be embedded in a biodegradable or soluble hydrogel. The drug loaded hydrogel is then placed inside the scaffold lumen to facilitate drug release. This approach may allow a larger amount of drug to be released to a target site.

After fabrication, a plurality of implants may be disposed in a single package which is then sealed. The implants may then undergo sterilization. A sealed, sterilized package may include 1 to 2 implants, 2 to 5 implants, 5 to 10 implants, 10 to 20 implants, 20 to 30 implants, 30 to 40 implants, or greater than 40 implants.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through movement. Tg of a given polymer can be dependent on the heating rate and can be influenced by the thermal history of the polymer as well as its degree of crystallinity. Furthermore, the chemical structure of the polymer heavily influences the glass transition by affecting mobility.

The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate.

The Tg of a polymer, unless otherwise specified, can refer to a polymer that is in a dry state or wet state. The wet state refers to a polymer exposed to blood, water, saline solution, or simulated body fluid. The Tg of the polymer in the wet state can correspond to soaking the polymer for at least 2 minutes or until it is saturated.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to a change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. The modulus typically is the initial slope of a stress-strain curve at low strain in the linear region.

### EXAMPLE

A transmyocardial revascularization procedure was performed on a swine by first ligating the porcine left anterior descending coronary artery (LAD) at the middle third of the artery to induce LAD occlusion and then inserting a drug loaded PLGA porous tubing into a drilled channel through the left ventricle wall of the swine. At six weeks post-operation, the implant group with heparin and bFGF promoted neovascular formation, enhanced blood-flow perfusion, and improved myocardial function.

Any combination of the features and embodiments described above is herein disclosed.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. A method of treating insufficient blood flow to a heart muscle comprising:
creating a channel in a heart muscle of a patient in need of increased blood flow to the heart muscle due to insufficient blood flow to the heart muscle; and
disposing an implant within the channel;
wherein the implant supports and maintains at least a portion of the channel to allow oxygen rich blood to flow through the channel.

2. The method of claim 1, wherein the implant comprises a shape defined by a wall that encloses a cavity or lumen.

3. The method of claim 1, wherein the implant comprises a tubular body comprising walls surrounding a lumen through which the blood flows.

4. The method of claim 1, wherein the implant comprises atubular body comprising walls surrounding a lumen, wherein the walls are fenestrated, porous, contain pores, or have open cells.

5. The method of claim 1, wherein the implant is bioresorbable and completely resorbs away from the channel after providing the support to the portion of the channel.

6. The method of claim 1, wherein the implant comprises a bioresorbable polymer.

7. A method of treating insufficient blood flow to a heart muscle comprising:
creating a channel in a heart muscle of a patient in need of increasing blood flow to the heart muscle due to insufficient blood flow to the heart muscle; and
disposing an implant within the channel, wherein the implant comprises an antithrombotic or anticoagulant active agent that reduces or prevents thrombosis in the channel and/or a growth factor active agent that promotes angiogenesis and growth of new capillaries in the heart muscle that provide additional blood to the heart muscle which alleviates the insufficient blood flow to the heart muscle.

8. The method of claim 7, wherein the antithrombotic or anticoagulant active agent is selected from the group consisting of sodium heparin, low molecular weight heparin, solvent soluble heparin such as TDMAC-heparin, benzalkonium heparin, fondaparinux, idraparinus, Xa inhibitor, coumadins, hirudin and its derivatives, EDTA and any combination thereof.

9. The method of claim 7, wherein the growth factor comprises basic fibroblast growth factor (bFGF), acidic FGF, vascular endothelial growth factor, platelet derived growth factor, stem cells, and any combination thereof.

10. The method of claim 7, wherein the implant comprises a tubular body comprising walls surrounding a lumen through which the blood flows.

11. The method of claim 7, wherein the implant comprises a tubular body comprising walls surrounding a lumen, wherein the walls are fenestrated, porous, contain pores, or have open cells.

12. The method of claim 7, wherein the implant is bioresorbable and completely resorbs away after releasing the active agent.

13. The method of claim 7, wherein the implant comprises a bioresorbable polymer.

14. The method of claim 7, wherein the implant comprises a coating including the active agent.

15. A method of treating insufficient blood flow to a heart muscle comprising:
creating a channel in a heart muscle of a patient in need of increased blood flow to the heart muscle due to insufficient blood flow to the heart muscle; and
disposing a hollow elongate implant within the channel, wherein bioresorbable structure is disposed within the hollow elongate implant and prevents blood flow through the hollow elongate implant;
wherein the bioresorbable implant comprises at least one active agent that are released in the heart muscle while blood flow is prevented,
wherein after a period of release of the at least one active agent, bioresorption of the structure allows blood flow through the implant.

16. The method of claim 15, wherein the at least one active agent comprises an effective amount of growth factor that promotes angiogenesis and growth of new capillaries in the heart muscle that provides additional blood to the heart muscle which alleviates the insufficient blood flow to the heart muscle.

17. The method of claim 15, wherein the implant comprises a hollow elongate body comprising walls surrounding a lumen through which the blood flows.

18. The method of claim 15, wherein the implant comprises a hollow elongate body comprising walls surrounding a lumen, wherein the walls are fenestrated, porous, contain pores, or have open cells.

19. The method of claim 15, further comprising radially expanding the hollow elongate implant after being disposed in the channel to an outer diameter larger than a diameter of the channel which provides for increased blood flow.

20. The method of claim 15, wherein the implant is bioresorbable and completely resorbs away after releasing the active agent.

21. The method of claim 15, wherein the implant comprises a bioresorbable polymer.

22. The method of claim 15, wherein the implant comprises a coating including the active agent.

23. An implant for treating insufficient blood flow to a heart muscle comprising:
a hollow elongate body comprising walls around a lumen, wherein the hollow elongate body comprises a bioresorbable polymer, wherein upon implantation in a channel in a heart muscle the hollow elongate body supports the channel which provides increased blood flow to the heart muscle;
an effective amount of an antithrombotic or anticoagulant active agent that reduces or prevents thrombotic closure of the channel; and
an effective amount of a growth factor active agent that promotes angiogenesis and growth of new capillaries in the heart muscle that provides additional blood to the heart muscle which alleviates the insufficient blood flow to the heart muscle.

24. The implant of claim 23, wherein the hollow elongate body is tubular and has an inside diameter of 1 to 2 mm.

25. The implant of claim 23, wherein the hollow elongate body is a radially expandable scaffold that is capable of being radially expanded at 37° C.

26. The implant of claim 23, wherein the hollow elongate body comprises a coating including a polymer and the antithrombotic or anticoagulant active agent.

27. The implant of claim 23, wherein the hollow elongate body comprises a coating including a polymer and the growth factor active agent.

28. The implant of claim 23, wherein the antithrombotic or anticoagulant active agent is disposed on an inner surface of the hollow elongate body and the growth factor active agent is disposed on an outer surface of the hollow elongate body.

29. An implant for treating insufficient blood flow to a heart muscle comprising:
a hollow elongate body comprising walls around a lumen, wherein the hollow elongate body is made of a bioresorbable polymer; and
a bioresorbable sponge inside the hollow elongate body, wherein the sponge contains an effective amount of antithrombotic or anticoagulant active agent and an effective amount of growth factor active agent(s) that promotes angiogenesis and growth of new capillaries in the heart muscle that provides additional blood to the heart muscle which alleviates the insufficient blood flow to the heart muscle when and after the polymers are degraded away.

30. The method of claim 29, wherein the walls of the hollow elongate body contain multiple holes.
